(19) United States
(12) Patent Application Publication
(10) Pub. No.: US 2010/0249634 A1
(43) Pub. Date: Sep. 30, 2010

(54) Mouthpiece and Airway Congestion Monitoring System

(76) Inventor: Leland G. Hansen, St. Paul, MN (US)

Correspondence Address: BRIGGS AND MORGAN P.A., 2200 IDS CENTER, 80 SOUTH 8TH ST, MINNEAPOLIS, MN, 55402, US

(21) Appl. No.: 12/728,193

(22) Filed: Mar. 19, 2010

Related U.S. Application Data
(60) Provisional application No. 61/161,707, filed on Mar. 19, 2009.

(51) Int. Cl. A61B 5/087; A61B 5/097

(52) U.S. Cl. 600/538; 600/543

(57) ABSTRACT

A congestion monitoring system includes a generally cylindrical, hollow form to channel a patient's breath, a flow restriction within the hollow form, and a pair of air ports. One of the air ports is located on the hollow form at a first side of the flow restriction, and the other air port is located on the hollow form at a second side of the flow restriction. The system also includes a coupling for connecting the pair of air ports to a pair of air lines, a sensor in communication with the air ports via the pair of air lines, and a monitor device. The monitor device converts a pressure signal from the air ports into a signal representative of a flow rate of the patient's breath through the hollow form.

### RELATED APPLICATIONS

This application claims priority under 35 U.S.C. §119(e) to U.S. Provisional Patent Application No. 61/161,707, filed Mar. 19, 2009, the contents of which are incorporated herein by reference.

### TECHNICAL FIELD

The present disclosure is generally directed to airflow measurement and more specifically to devices and methods for measuring patient airflow and evaluating pulmonary function.

### BACKGROUND OF THE INVENTION

Many guidelines pertaining to the treatment of patients with asthma and chronic obstructive pulmonary disease (COPD) state that it is important to use pulmonary function tests to evaluate the lung function of patients. See Joost J. den Otter et al., Lung Function Measurement in General Practice: General Practice Measurements Compared with Laboratory Measurements During the DIMCA Trial, 17 Family Practice 314 (2000), said reference being incorporated by reference herein. Unfortunately, it remains difficult for a patient to evaluate levels of airway congestion without access to diagnostic tools of a pulmonary function laboratory. Obtaining access to such tools is both time-consuming and expensive.

A need exists for a portable, reasonably accurate tool for evaluating levels of airway congestion of a patient.

A need also exists for a device and system for evaluating trends of lung congestion over time.

### SUMMARY OF THE INVENTION

The present invention provides a mouthpiece and method of using same within a congestion monitoring system. The mouthpiece can be coupled to a pressure monitoring device via a pair of air lines. The mouthpiece, via an internal flow restricting structure, establishes a pressure differential which is communicated to the monitoring device. During patient expiration, the mouthpiece functions to accurately and consistently provide a pressure differential to the monitor for conversion into a patient-usable format. One aspect of the monitoring device is the provision of statistical analyses of stored measurements, for example to provide a trend analysis and report of the information for the patient.

The present invention is directed to a congestion monitoring system including a generally cylindrical, hollow form to channel a patient's breath, a flow restriction within the hollow form, and a pair of air ports. One of the air ports is located on the hollow form at a first side of the flow restriction, and the other air port is located on the hollow form at a second side of the flow restriction. The system also includes a coupling for connecting the pair of air ports to a pair of air lines, a sensor in communication with the air ports via the pair of air lines, and a monitor device. The monitor device converts a pressure signal from the air ports into a signal representative of a flow rate of the patient's breath through the hollow form. The monitor may also provide a comparison over time of the air flow of the patient, to establish whether the patient's lung function is stable.

The present invention is also directed to a congestion monitoring system comprising a mouthpiece including a generally cylindrical, hollow form to channel a patient's breath. The mouthpiece also includes a flow restriction within the hollow form and a pair of air ports. One of the air ports is located on the hollow form at a first side of the flow restriction, and the other air port is located on the hollow form at a second side of the flow restriction. The system also includes a pair of air lines coupled to the pair of air ports, a sensor in communication with the pair of air lines, and a monitor device. The sensor converts a pressure signal from the pair of air lines into an electrical signal which is provided to the monitor device.

The present invention is further directed to a method of providing information relating to pulmonary function. The method includes providing a mouthpiece to a patient. The provided mouthpiece includes a generally cylindrical, hollow form to channel the patient's breath. The mouthpiece also includes a flow restriction within the hollow form and a pair of air ports. One of the air ports is located on the hollow form at a first side of the flow restriction, and the other air port is located on the hollow form at a second side of the flow restriction. The method further includes coupling the pair of air ports to a pair of air lines, receiving a pressure signal from the air lines at a sensor for a predetermined period of time, converting the pressure signal into an electrical signal representative of a flow rate of the patient, and transmitting the electrical signal to a monitor device.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings, in which:

FIG. 1 depicts an embodiment of a congestion monitoring system in accordance with the present invention.

FIG. 2 is a graph of airflow in liters per second versus volts (of a differential pressure sensor) for an experiment using a mouthpiece in accordance with the present invention.

FIG. 3 is a graph of measured airflow versus calculated air flow for an experiment using a congestion monitoring system of the present invention.

FIG. 4 is a perspective view of an embodiment of a mouthpiece for use in a system in accordance with the present invention.

FIG. 5 is a perspective view of the mouthpiece of FIG. 4 attached to a pair of air lines.

FIG. 6a is a top view of the mouthpiece of FIG. 4. FIG. 6b is a side view of the mouthpiece of FIG. 4. FIG. 6c is a bottom view of the mouthpiece of FIG. 4.

FIG. 7 is a cross sectional view of the mouthpiece of FIG. 4.

FIG. 8 is a detailed cross sectional view of a portion of the mouthpiece of FIG. 4 as indicated by Detail A in FIG. 7.

FIG. 9 is a detailed cross sectional view of a portion of the mouthpiece of FIG. 4 as indicated by Detail B in FIG. 7.

### DETAILED DESCRIPTION OF THE INVENTION

A congestion monitor in accordance with the present invention is designed to measure total volume of air expired in the first second of a forced expiratory breath. This value when monitored on a regular basis can be a valuable tool in managing chronic obstructive pulmonary diseases. It is often difficult for a patient to evaluate the gradual decline of his/her lung function without pulmonary testing over time. Using the congestion monitor of the present invention on a regular basis can indicate to the patient whether his/her lung function is stable, decreasing or improving. A congestion monitor in accordance with the present invention provides the patient the opportunity to better evaluate combinations of therapy, for example whether or not to increase therapy, and/or to contact his/her physician.

The congestion monitor is designed to measure, with consistency, a one second volume of air flow. This measurement is repeatable to within ±3% over a range of 0 to 12 liters per second. The consistency of measurement using the system of the present invention allows the patient to establish a base line measurement that can be used to show trending of lung congestion over time. The congestion monitor of the present invention does not provide a measure of true FEV1 (“forced expiratory volume in 1 second”) values and should not be compared to FEV1 values measured in a pulmonary function laboratory.

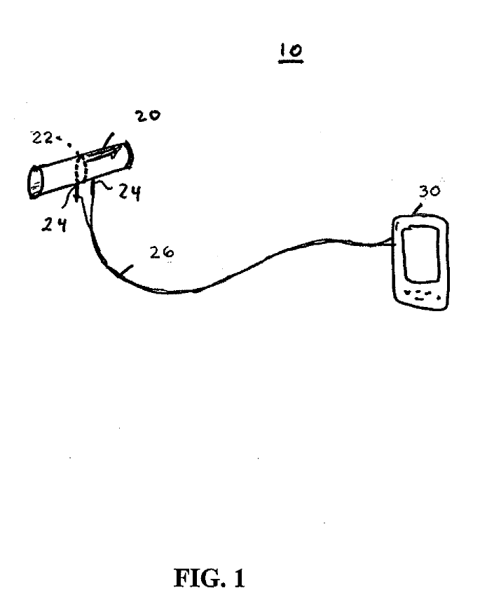

FIG. 1 depicts a congestion monitoring system 10 in accordance with the present invention. System 10 includes a mouthpiece 20 adapted to be inserted into or cover a patient's mouth during expiration. Mouthpiece 20 is a hollow, generally cylindrical form including a flow restricting structure, such as a ring 22, inside the mouthpiece 20. A pair of air ports 24 are provided on either side of the flow restricting structure. The air ports 24 are coupled via air lines 26 to a congestion monitor 30. Alternatively, air lines 26 can be attached to an intermediate sensor (not shown) for converting air pressure into an analog or digital signal which can be communicated to monitor device 30. Flow restricting structure, ring 22, interrupts the flow of air through the mouthpiece 20 and establishes a pressure differential between the pair of air ports 24. The pressure differential information is communicated (via analog or digital communication or both) to monitor 30 via air lines 26.

Monitor 30 may include a variety of displays relating to measured values, previous values, desired values, etc. Monitor 30 may include software for performing a variety of different evaluations on the data collected, including but not limited to trend analyses.

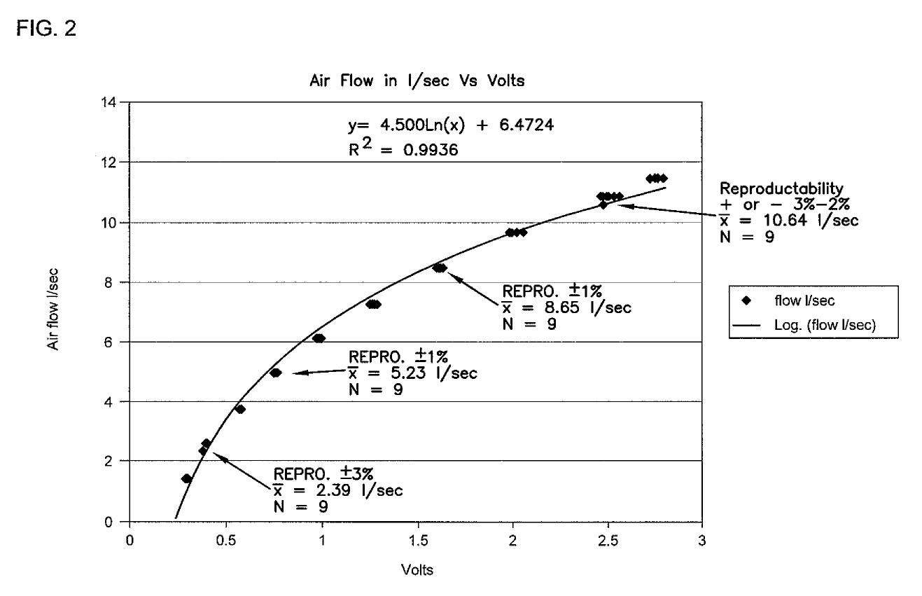

FIG. 2 is a graph of airflow in liters per second versus volts (of a differential pressure sensor) for an experiment using a mouthpiece in accordance with the present invention, as described above. As shown in FIG. 2, the mouthpiece yields accurate and consistent measurements over the 0-12 liters per second range. For the mouthpiece used in the experiment of FIG. 2, the inner diameter of the hollow form was 2.2 cm and the outer diameter was 2.54 cm. The diameter inside the ring 22 of the mouthpiece was 1.6 cm.

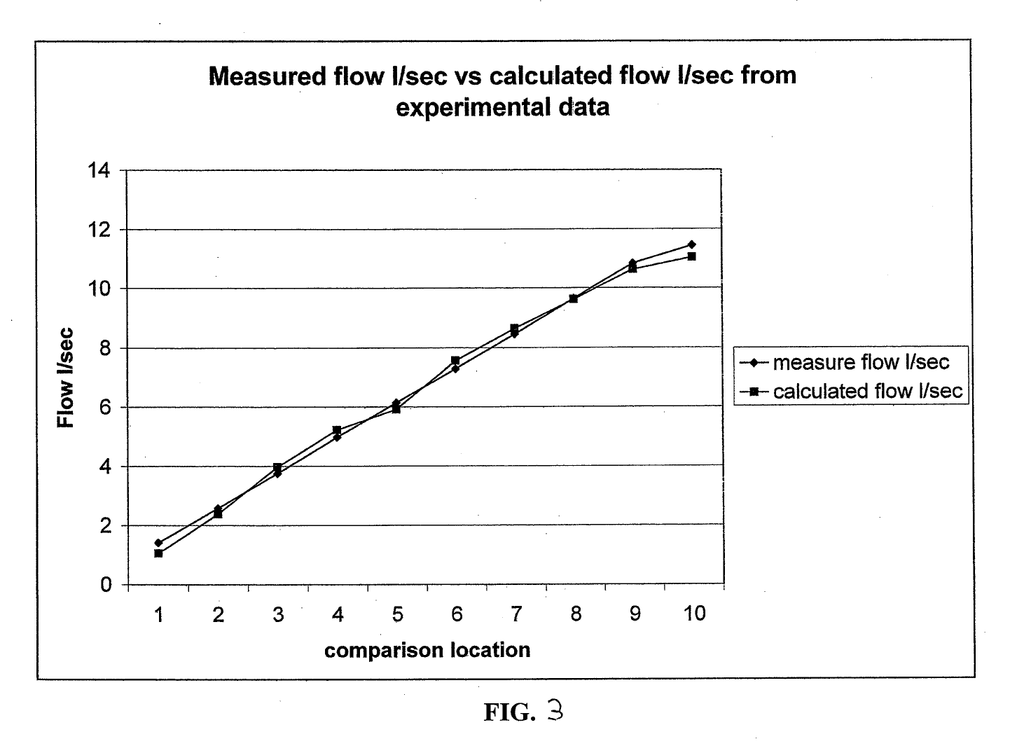

FIG. 3 is a graph of measured airflow versus calculated air flow for the experiment. FIG. 3 demonstrates a desired high accuracy of the regression equation for calculating flows from 0-12 liters per second.

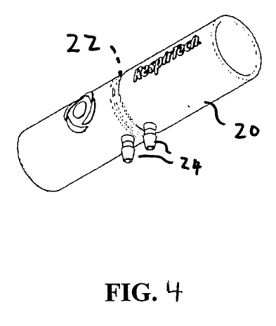

FIG. 4 is a perspective illustration of another embodiment of a mouthpiece 20 for application in a system in accordance with the present invention. Mouthpiece 20 is a hollow, generally cylindrical form including a flow restricting structure, such as a ring 22, inside the mouthpiece 20. A pair of air ports 24 are provided on either side of the flow restricting structure.

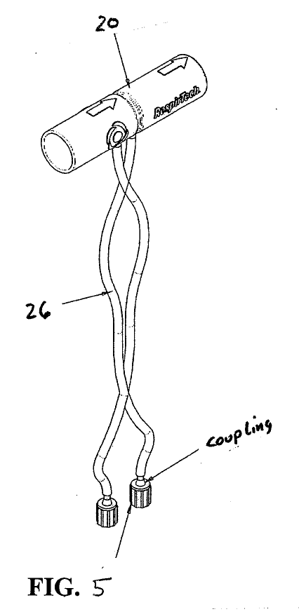

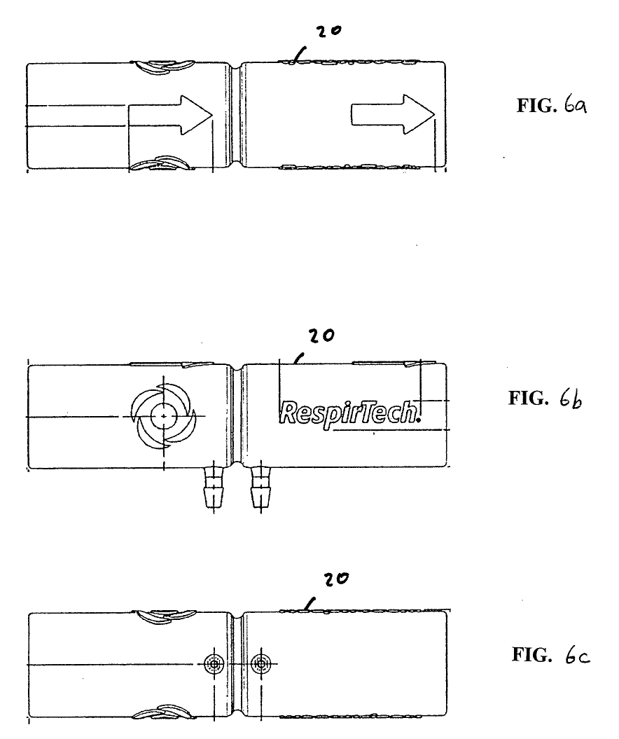

FIG. 5 is a perspective illustration of the mouthpiece 20 of FIG. 4 attached to a pair of air lines 26 via air ports 24. The ends of the air lines 26 opposite from the ends coupled to the mouthpiece are adapted to be coupled to a congestion monitor device. FIGS. 6a, 6b, and 6c illustrate a top view, a side view, and a bottom view of the mouthpiece of FIG. 4, respectively.

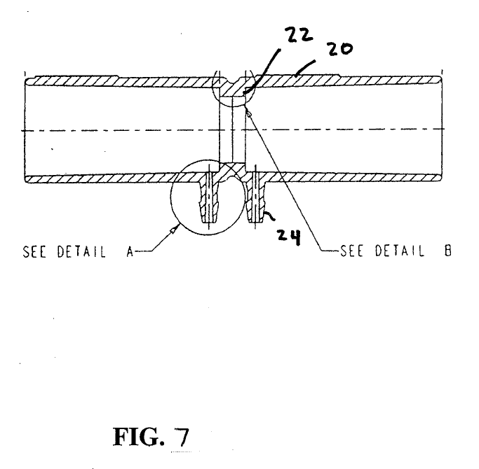

FIG. 7 is a cross sectional view of the mouthpiece of FIG. 4. In the embodiment shown, the inner diameter of the hollow form is 2.2 cm and the outer diameter is 2.54 cm. The diameter inside the ring 22 of the mouthpiece is 1.6 cm. The length of the hollow form is 5.08 cm, and the width of ring 22 along the hollow form is 6.3 mm. The air ports 24 have an inner diameter of 1.5 mm and an outer diameter of 2.5 mm. The walls of the hollow form taper at each end of the mouthpiece.

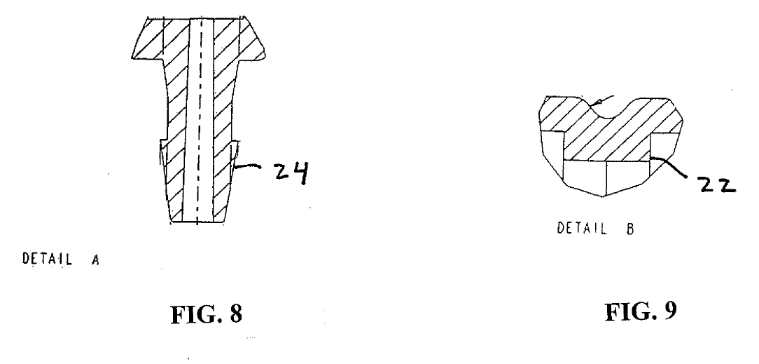

FIG. 8 is a detailed cross sectional view of a portion of the mouthpiece of FIG. 4 as indicated by Detail A in FIG. 7. FIG. 9 is a detailed cross sectional view of a portion of the mouthpiece of FIG. 4 as indicated by Detail B in FIG. 7.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A congestion monitoring system comprising:
a generally cylindrical, hollow form to channel a patient's breath;
a flow restriction within the hollow form;
a pair of air ports, wherein the first air port of the pair of air ports is located on the hollow form at a first side of the flow restriction, and the second air port of the pair of air ports is located on the hollow form at a second side of the flow restriction; and
a coupling for connecting the pair of air ports to a pair of air lines;
a sensor in communication with the air ports via the pair of air lines; and
a monitor device converting a pressure signal from said air ports into a signal representative of a flow rate of said patient's breath through said hollow form, and said monitor providing a comparison over time of air flow of said patient to establish whether a lung function of said patient is stable.

2. The congestion monitoring system of claim 1 wherein the flow restriction is a ring form.

3. The congestion monitoring system of claim 2 wherein the ring is positioned generally midway between a pair of ends of the hollow form.

4. A congestion monitoring system comprising:
a mouthpiece comprising a generally cylindrical, hollow form to channel a patient's breath, said mouthpiece including a flow restriction within the hollow form and a pair of air ports, wherein the first air port of the pair of air ports is located on the hollow form at a first side of the flow restriction, and the second air port of the pair of air ports is located on the hollow form at a second side of the flow restriction;
a pair of air lines coupled to the pair of air ports;
a sensor in communication with the pair of air lines; and
a monitor device, wherein the sensor is adapted to convert a pressure signal from the pair of air lines into an electrical signal which is provided to the monitor device.

5. The congestion monitoring system of claim 4 wherein the sensor is a differential pressure sensor.

6. The congestion monitoring system of claim 4 wherein the sensor is located within the monitor device

7. The congestion monitoring system of claim 4 wherein the monitor device is adapted to calculate an airflow rate based on said pressure signal.

8. The congestion monitoring system of claim 7 wherein the monitor device determines a trend analysis of airflow rates of said patient.

9. The congestion monitoring system of claim 8 wherein the monitor device provides the trend analysis as a visual display of airflow rates of said patient over time, said visual display indicating whether the a lung function of said patient is decreasing or increasing.

10. A method of providing information relating to pulmonary function comprising:
providing a mouthpiece to a patient, said mouthpiece comprising a generally cylindrical, hollow form to channel a breath of the patient, said mouthpiece including a flow restriction within the hollow form and a pair of air ports, wherein the first air port of the pair of air ports is located on the hollow form at a first side of the flow restriction, and the second air port of the pair of air ports is located on the hollow form at a second side of the flow restriction;
coupling the pair of air ports to a pair of air lines;
receiving a pressure signal from the air lines at a sensor for a predetermined period of time;
converting the pressure signal into an electrical signal representative of a flow rate of said patient; and
transmitting the electrical signal to a monitor device.

11. The method of claim 10 wherein the wherein the predetermined period of time is less than approximately 3 seconds.

12. The method of claim 10 wherein the predetermined period of time is approximately 1 second.

13. The method of claim 10, further comprising calculating an airflow rate based on the electrical signal.

14. The method of claim 13, further comprising calculating a volume of airflow based on the electrical signal.

15. The method of claim 14, further comprising performing a trend analysis based at least in part on said volume of airflow or said airflow rate.

16. The method of claim 15, further comprising displaying the volume of airflow or said airflow rate on the monitor device.
